(19) United States
(12) Patent Application Publication
(10) Pub. No.: US 2002/0019346 A1
(43) Pub. Date: Feb. 14, 2002

(54) Treatment of prostate cancer by inhibiting lyn tyrosine kinase

(75) Inventor: Shmuel A. Ben-Sasson, Jerusalem (IL)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Children's Medical Center Corporation, Boston, MA (US)

(21) Appl. No.: 09/735,279

(22) Filed: Dec. 11, 2000

Related U.S. Application Data
(63) Continuation-in-part of application No. 08/861,153, filed on May 21, 1997.

(51) Int. Cl. A61K 48/00; A61K 38/17; A61K 31/519; A61K 39/395

(52) U.S. Cl. 514/12; 514/44; 424/155.1; 514/256; 514/258

(57) ABSTRACT

Prostate cancer can be treated by administering one or more inhibitros of lyn tyrosine kinase to an individual who has or is susceptible to contracting prostate cancer. The inhibitors include peptides containing between six and eleven amino acids.

### RELATED APPLICATIONS

This application is a continuation-in-part of International Application No. PCT/US98/10321, filed May 20, 1998, which is a continuation-in-part of U.S. application Ser. No.: 08/861,153 filed on May 21, 1997. The entire teachings of the above applications are incorporated herein by reference.

### BACKGROUND OF THE INVENTION

Protein tyrosine kinases are members of the eukaryotic protein kinase superfamily. Enzymes of this class specifically phosphorylate tyrosine residues of intracellular proteins and are important in mediating signal transduction in multicellular organisms. Protein tyrosine kinases occur as membrane-bound receptors, which participate in transmembrane signaling, or as intracellular proteins which take part in signal transduction within the cell, including signal transduction to the nucleus.

As such, phosphorylation of tyrosine by protein tyrosine kinases is an important mechanism for regulating intracellular events in response to environmental changes. A wide variety of cellular events, including cytokine responses, antigen-dependent immune responses, cellular transformation by RNA viruses, oncogenesis, regulation of the cell cycle and modification of cell morphology and phenotype are regulated by protein tyrosine kinases.

Enhanced protein tyrosine kinase activity can lead to persistent stimulation by secreted growth factors, for example, which, in turn, can lead to proliferative diseases such as cancer, to nonmalignant proliferative disease such as arteriosclerosis, psoriasis and to inflammatory response such as septic shock.

Src is among the first protein kinases described whose uncontrolled expression is directly linked to malignant transformation. The Src family contains several members. Some of these members are ubiquitously expressed while others like Lck, Hck, and Lyn have been, until recently, thought to be expressed primarily in cells of the immune system.

Prostate cancer is a malignancy with high incidence in many countries. It is also a cancer with high morbidity and mortality rates and constitutes one of the leading causes of death or disability among males. Its early detection and eradication is aggressively pursued world wide.

There exists a need for further methods to alleviate and eliminate prostate cancer. In particular, there exists a need for substances which can be administered to individuals who have or are susceptible to developing prostate cancer. In addition, there is a need for further information regarding the role of protein kinases in malignant transformation and for substances which interact with these substances in such a manner that the malignant transformation is reversed.

### SUMMARY OF THE INVENTION

The invention relates to methods of treating prostate cancer in individuals by administering one or more inhibitors of lyn tyrosine kinase to these individuals. The administration of inhibitors of lyn tyrosine kinase causes a reduction in prostate cancer growth in those individuals. The reduction in prostate cancer growth ranges from a diminution in the prostate cancer growth rate, when compared to the cancer growth without lyn tyrosine kinase inhibitor administration, to stability in size or shrinkage of the tumor and, in the best cases, to elimination of the tumor.

Any inhibitor of lyn tyrosine kinase can be administered to the individuals in the course of treating prostate cancer. Among the lyn tyrosine kinase inhibitors that can be employed are peptides, antibodies immunoreactive with lyn tyrosine kinases, anti-sense nucleic acids that block expression of lyn tyrosine kinases, negative dominant Lyn tyrosine kinase genes which express lyn tyrosine kinase proteins with reduced or nonexistent biological activity, and small organic molecules. Any of these inhibitors of lyn tyrosine kinase will inhibit the growth of prostate cancer in individuals.

Preferred inhibitors of lyn tyrosine kinase, which cause a reduction in prostate cancer growth, are peptides. Particularly preferred inhibitors of lyn tyrosine kinase are the peptides designated as K055H007 (SEQ ID NO:1); K055H101 (SEQ ID NO:2); K055H104 (SEQ ID NO:3); K055H108 (SEQ ID NO:4); K055H110 (SEQ ID NO:5); K055H111 (SEQ ID NO:6); K055H112 (SEQ ID NO:7); K055H113 (SEQ ID NO:8); K055H114 (SEQ ID NO:9); K055H115 (SEQ ID NO:10); K055H116 (SEQ ID NO: 11); K055H117 (SEQ ID NO: 12); K055H118 (SEQ ID NO:13); K055Hl 19 (SEQ ID NO:14); K055H120 (SEQ ID NO:15); K055H121 (SEQ ID NO:16); K055H122 (SEQ ID NO:17); K055H123 (SEQ ID NO:18); K055H124 (SEQ ID NO:19); K055H125 (SEQ ID NO:20); K055H129 (SEQ ID NO:21); K055H130 (SEQ ID NO:22); K055H134 (SEQ ID NO:23); K055H135 (SEQ ID NO:24); K055H136 (SEQ ID NO:25); K055H137 (SEQ ID NO:26); K055H138 (SEQ ID NO:27); K055H139 (SEQ ID NO:28); K055H140 (SEQ ID NO:29); K055H142 (SEQ ID NO:30); K055H143 (SEQ ID NO:31); K055H144 (SEQ ID NO:32); K055H145 (SEQ ID NO:33); K055H146 (SEQ ID NO:34); K055H147 (SEQ ID NO:35); K055H148 (SEQ ID NO:36); K055H149 (SEQ ID NO:37); K055H152 (SEQ ID NO:38); K055H153 (SEQ ID NO:39); K055H154 (SEQ ID NO:40); K055H155 (SEQ ID NO:41); K055H161 (SEQ ID NO:42); K055H162 (SEQ ID NO:43); K055H163 (SEQ ID NO:44); K055H164 (SEQ ID NO:45); K055H165 (SEQ ID NO:46); K055H166 (SEQ ID NO:47); K055H167 (SEQ ID NO:48); K055H168 (SEQ ID NO:49); K055H169 (SEQ ID NO:50); K055H170 (SEQ ID NO:51); K055H171 (SEQ ID NO:52); K055H172 (SEQ ID NO:53); K055H173 (SEQ ID NO:54); K055H174 (SEQ ID NO:55); K055H175 (SEQ ID NO:56); K055H176 (SEQ ID NO:57); K055H177 (SEQ ID NO:58); K055H300 (SEQ ID NO:59); K055H301 (SEQ ID NO:60); K055H302 (SEQ ID NO:61); K055H304 (SEQ ID NO:62); K055H305 (SEQ ID NO:63); K055H306 (SEQ ID NO:64); K055H307 (SEQ ID NO:65); K055H801 (SEQ ID NO:66); K055H902 (SEQ ID NO:67); K055H908 (SEQ ID NO:68); K055H910 (SEQ ID NO:69); K055H911 (SEQ ID NO:70); K055H912 (SEQ ID NO:71); K055H919 (SEQ ID NO:72); K055H923 (SEQ ID NO:73); K055H925 (SEQ ID NO:74), as specified in FIG. 2. These peptides, and derivatives of these peptides that retain their ability to inhibit lyn tyrosine kinase, are themselves embodiments of the present invention.

This invention also relates to the inhibition of the growth of prostate cancer cells by administering one or more inhibitors of lyn tyrosine kinase to the prostate cancer cells. The administration of inhibitors of lyn tyrosine kinase to prostate cancer cells causes a retardation in the growth of these cells and, at least eventually, causes a reduction in the number of these cells. Again, any inhibitor of lyn tyrosine kinase will inhibit the growth of prostate cancer cells when delivered to these cells. The inhibitors include peptides, antibodies, anti-sense nucleic acids, negative dominant Lyn tyrosine kinase genes, and small organic molecules. Exemplary inhibitors are peptides and derivatives of these peptides that retain inhibitory activity of lyn tyrosine kinase, that are set forth within this application.

The peptides of the present invention, in addition to their ability to reduce prostate cancer growth in individuals or their ability to inhibit the growth of prostate cancer cells, also are useful for generating antibodies that reduce prostate cancer growth and inhibit the growth of prostate cancer cells. The peptides act as antigenic agents for producing such antibodies. These antibodies, in turn, act as inhibitors of lyn tyrosine kinase, thereby reducing prostate cancer growth and inhibiting prostate cancer cell growth when they are administered to the individual with prostate cancer or to the prostate cancer cells.

### BRIEF DESCRIPTION OF THE DRAWINGS

FIG. 1 is a Table illustrating the amino acid sequences of the lyn peptides.

FIGS. 2A-2B is a Table illustrating the sequences of the peptides K055H007 (SEQ ID NO:1); K055H101 (SEQ ID NO:2); K055H104 (SEQ ID NO:3); K055H108 (SEQ ID NO:4); K055H110 (SEQ ID NO:5); K055H111 (SEQ ID NO:6); K055H112 (SEQ ID NO:7); K055H113 (SEQ ID NO:8); K055H114 (SEQ ID NO:9); K055H115 (SEQ ID NO:10); K055H116 (SEQ ID NO:11); K055H117 (SEQ ID NO: 12); K055H118 (SEQ ID NO:13); K055H119 (SEQ ID NO:14); K055H120 (SEQ ID NO:15); K055H121 (SEQ ID NO:16); K055H122 (SEQ ID NO:17); K055H123 (SEQ ID NO:18); K055H124 (SEQ ID NO:19); K055H125 (SEQ ID NO:20); K055H129 (SEQ ID NO:21); K055H130 (SEQ ID NO:22); K055H134 (SEQ ID NO:23); K055H135 (SEQ ID NO:24); K055H136 (SEQ ID NO:25); K055H137 (SEQ ID NO:26); K055H138 (SEQ ID NO:27); K055H139 (SEQ ID NO:28); K055H140 (SEQ ID NO:29); K055H142 (SEQ ID NO:30); K055H143 (SEQ ID NO:31); K055H144 (SEQ ID NO:32); K055H145 (SEQ ID NO:33); K055H146 (SEQ ID NO:34); K055H147 (SEQ ID NO:35); K055H148 (SEQ ID NO:36); K055H149 (SEQ ID NO:37); K055H152 (SEQ ID NO:38); K055H153 (SEQ ID NO:39); K055H154 (SEQ ID NO:40); K055H155 (SEQ ID NO:41); K055H161 (SEQ ID NO:42); K055H162 (SEQ ID NO:43); K055H163 (SEQ ID NO:44); K055H164 (SEQ ID NO:45); K055H165 (SEQ ID NO:46); K055H166 (SEQ ID NO:47); K055H167 (SEQ ID NO:48); K055H168 (SEQ ID NO:49); K055H169 (SEQ ID NO:50); K055H170 (SEQ ID NO:51); K055H171 (SEQ ID NO:52); K055H172 (SEQ ID NO:53); K055H173 (SEQ ID NO:54); K055H174 (SEQ ID NO:55); K055H175 (SEQ ID NO:56); K055H176 (SEQ ID NO:57); K055H177 (SEQ ID NO:58); K055H300 (SEQ ID NO:59); K055H301 (SEQ ID NO:60); K055H302 (SEQ ID NO:61); K055H304 (SEQ ID NO:62); K055H305 (SEQ ID NO:63); K055H306 (SEQ ID NO:64); K055H307 (SEQ ID NO:65); K055H801 (SEQ ID NO:66); K055H902 (SEQ ID NO:67); K055H908 (SEQ ID NO:68); K055H910 (SEQ ID NO:69); K055H911 (SEQ ID NO:70); 055H912 (SEQ ID NO:71); K055H919 (SEQ ID NO:72); K055H923 (SEQ ID NO:73); K055H925 (SEQ ID NO:74).

FIG. 3 is a graph showing the percent inhibition of proliferation of DU145 prostate cancer cells by increasing concentrations (μM) of K055H101 (SEQ ID NO.:2).

FIG. 4 is a graph showing the percent inhibition of proliferation of PC3 prostate cancer cells by increasing concentrations (EM) of K055H101(SEQ ID NO.:2) relative to control cells.

FIGS. 5A-5F are graphs showing the percent inhibition of proliferation of DU145 prostate cancer cells by increasing concentrations (EM) of a number of lyn peptides.

FIG. 6 is a graphical representation of the percentage change of prostate cancer tumor volume over a period of time for control animals and a group of animals to whom the peptide K055H137 (tbi) (SEQ ID NO:19) had been administered.

### DETAILED DESCRIPTION OF THE INVENTION

A protein tyrosine kinase (hereinafter “PTK”) is a membrane bound or intracellular protein which uses the gamma phosphate of ATP or GTP to generate phosphate monoesters on the phenolic group of a tyrosine residue. PTKs have homologous “kinase domains”or “catalytic domains”or which carry out this phosphorylation.

This invention is directed to methods of treating prostate cancer by administering one or more inhibitors of lyn tyrosine kinase to individuals who have or are likely to develop prostate cancer. Therapeutically effective amounts of the inhibitor(s) are delivered to the individual. When these inhibitors are administered to those individuals who have prostate cancer, the growth of this cancer is diminished. In many instances, the prostate cancer growth ceases, i.e., stasis of the prostate cancer tumor occurs, and, in preferred cases, the prostate cancer tumor diminishes or is reduced in size. In most preferred cases, the prostate cancer tumor is totally eliminated. For prophylactic situations or for those individuals who are susceptible to contracting prostate tumor cancer, the administration of one or more inhibitors of lyn tyrosine kinase will reduce the likelihood of the individual contracting the disease. In preferred situations, the individual to whom the lyn tyrosine kinase inhibitor is administered does not contract the disease.

The reason that the administration of inhibitors of lyn tyrosine kinase to individuals with prostate cancer causes a reduction of prostate cancer growth or a reduction in prostate cancer tumor size is that it has been found that lyn tyrosine kinase is involved in the growth and viability of prostate cancer cells. The lyn tyrosine kinase is active in prostate cancer tumor cells. Inhibition of lyn tyrosine kinase in these cells causes a cessation in the activity of this tyrosine kinase. The cessation in activity in these cells results in the observed inhibition of prostate cancer cell or tumor growth. The administration of inhibitors of lyn tyrosine kinase is critically linked to the reduction in prostate cancer growth by the inhibition of the activity of lyn tyrosine kinase by these inhibitors in prostate cancer cells.

This invention is also directed to methods of inhibiting the growth of prostate cancer cells, whether within the body of an individual (who may or may not have prostate cancer) or anywhere outside an individual's body, such as in an in vitro setting. These methods are directed to administering one or more inhibitors of lyn tyrosine kinase to the prostate cancer cells. The inhibitor or inhibitors is (are) administered in amounts that are effective in inhibiting the growth of the prostate cancer cells. When the inhibitors are administered to the prostate cancer cells, the cells stop growing or dividing as rapidly as they did in the absence of the inhibitors. In many instances, growth of the prostate cancer cells entirely ceases. Quite often, the prostate cancer cells lose viability and die. The growth retardation or death of the prostate cancer cells occurs because lyn tyrosine kinase is involved with growth and viability of prostate cancer cells. When inhibitors of lyn tyrosine kinase are administered to prostate cancer cells, the activity of this kinase is severely retarded or ceased. This retardation or cessation of lyn tyrosine kinase activity results in the growth retardation or death of the prostate cancer cells.

Any inhibitor of lyn tyrosine kinase will work in the present invention. For example, low molecular weight organic molecules can act as inhibitors of lyn tyrosine kinase. Such low molecular weight organic molecules are known in the art. Exemplary of such compounds is the pyrazolo pyrimidine tyrosine kinase inhibitor PP1 (see Schindler et al. “Crystal Structure of Hck in Complex with a Src Family-Selective Tyrosine Kinase Inhibitor”, Molecular Cell, Vol. 3, 639-648, May 1999, the pertinent contents of which are incorporated herein by reference). Other organic compound inhibitors of the Src family tyrosine kinases are known. Preferred low molecular weight organic molecules for use with the present invention are those that specifically inhibit the activity of lyn tyrosine kinase.

Another type of inhibitor of lyn tyrosine kinase is anti-sense nucleic acids. The nucleic acids are single stranded ribonucleic or deoxyribonucleic acid strands which contain several (tens) nucleotides joined together through normal sugar-phosphate bonds. These strands bind via hybridization within or upstream of the structural gene, that encodes lyn tyrosine kinase. This hybridization binding blocks proper transcription or expression of lyn tyrosine kinase from occurring in prostate cancer cells. Since proper transcription or expression is effectively blocked by the hybridization of the antisense nucleic acids to the DNA or RNA that contains the lyn tyrosine kinase structural gene, inhibition of this kinase is effected. There is much less active lyn tyrosine kinase produced by the prostate cancer cells than without the presence of these anti-sense nucleic acids. This diminution of the amount of active lyn tyrosine kinase necessarily is manifested as an inhibition of lyn tyrosine kinase in the prostate cancer cells. The particular nucleotides that are joined together to form the anti-sense nucleic acids are those that are complementary to the region of the lyn tyrosine kinase structural gene. Thus, the anti-sense nucleic acids are complementary to the region of the lyn tyrosine kinase to which the anti-sense nucleic acids bind via hybridization. These nucleotides of the anti-sense nucleic acids are specifically determined by the nucleotides of the target location and can easily be identified by the skilled practitioner once the sequence of the target location is established. The target location is a matter of choice to some extent. It lies within the region of the structural gene that encodes lyn tyrosine kinase or is upstream of this coding region in the recognition or regulation region of the lyn tyrosine kinase gene. The target location nucleotide sequence can easily be established by the skilled practitioner from publicly available information concerning the lyn tyrosine kinase gene or can be obtained by routine examination of homologous genes coupled with standard molecular biology techniques.

Still another type of inhibitor of lyn tyrosine kinase is negative dominant Lyn tyrosine kinase genes. The presence of these genes in prostate cancer cells allows nonfunctional lyn tyrosine kinase to be expressed to the exclusion of functional lyn tyrosine kinase. The negative dominant lyn tyrosine kinase in the prostate cancer cells is inhibitory of lyn tyrosine kinase activity because this kinase is non-functional. Nonfunctional kinases, by definition, have no kinase activity. Negative dominant Lyn tyrosine kinase genes are introduced into prostate cancer cells by gene transfer techniques, which are becoming increasingly more standard in the art (calcium precipitation, electrical discharge, physical injection, use of carriers such as recombinant vectors, etc.). The introduced negative dominant Lyn tyrosine kinase gene is incorporated in the prostate cancer cell genome. There, copies of it are passed to progeny cells. Since this Lyn tyrosine kinase gene is negative dominant, it will be expressed in response to signals which induce lyn tyrosine kinase expression rather than the active form of lyn tyrosine kinase. Prostate cancer cells which have incorporated the negative dominant Lyn tyrosine kinase gene will not grow because the expressed lyn tyrosine kinase is inactive. The negative dominant Lyn tyrosine kinase genes can be found in the art or can be produced by standard gene mutation techniques which are well known to skilled practitioners in the art. These genes can be suitably packaged for transgenic procedures by appropriate methods and materials known to the skilled practitioners.

A further type of inhibition of lyn tyrosine kinase is antibodies that are immunoreactive with lyn tyrosine kinase. These antibodies bind to the kinase and thereby severely limit or prohibit its kinase activity. The antibodies can be of any class or type. The binding site of the antibodies can be anywhere on the lyn tyrosine kinase molecule provided the immunoreactive binding between the antibody and the kinase molecule results in a severe inhibition of lyn tyrosine kinase activity. The antibodies can be polyclonal or monoclonal and are produced by well-known techniques to the skilled practitioner by using the lyn tyrosine kinase or an immunogenic fragment thereof as the antigenic stimulus. The antibodies can be delivered to the prostate cancer cells by depositing suitable clonal cells, which produce the antibodies, into the individual who has prostate cancer or who is susceptible to contracting prostate cancer. These clonal cells secrete the antibodies into the bloodstream where they are carried to the prostate cancer cells for immunoreaction with the lyn tyrosine kinase molecules. Binding fragments of antibodies are also suitable provided they bind lyn tyrosine kinase with sufficient affinity that the activity of the kinase is at least severely limited. Alternatively, the antibodies or suitable binding fragments can be introduced into prostate cancer cells by any of a variety of techniques known to the skilled practitioner (physical injection, attachment to carriers that cross cell membranes, transgenic introduction into the prostate cancer cells for subsequent induction of expression, etc.). The secreted, introduced or expressed antibodies or suitable antibody fragments thereof immunoreactively bind to the lyn tyrosine kinase molecules, thereby inhibiting their activity.

A further type of inhibitor of lyn tyrosine kinase is peptides, which herein are designated as lyn-derived peptides. These peptides are the preferred embodiment of this invention as inhibitors of lyn tyrosine kinase and thereby as agents to retard the growth, reduce or eliminate prostate cancer in an individual. The peptides apparently bind to lyn tyrosine kinase and inhibit the activity of this kinase. This lyn tyrosine kinase inhibition causes a reduction in the growth of prostate cancer tumors. Quite often the tumors are reduced in size and many are eliminated. The peptides can be produced by a variety of techniques known to the skilled practitioner including organic synthetic procedures and production from cells that contain one or more genes that encode these peptides. These genes can be incorporated in the host cells by recombinant techniques.

Optionally, the C-terminus or the N-terminus of the peptides of the present invention, or both, can be substituted with a carboxylic acid protecting group or an amine protecting group, respectively. Suitable protecting groups are described in Green and Wuts, “Protecting Groups in Organic Synthesis”, John Wiley and Sons, Chapters 5 and 7, 1991, the teachings of which are incorporated herein by reference. Preferred protecting groups are those that facilitate transport of the peptide into a cell, for example, by reducing the hydrophilicity and increasing the lipophilicity of the peptide. In addition, a modified lysine residue can be added to the carboxy terminus to enhance biological activity. Examples of the lysine modification include the addition of an aromatic substitute, such as benzoyl, or an aliphatic group, such as acyl, or a myristic or stearic acid, at the epsilon amino group of the lysine residue. Examples of N-terminal protecting groups include acyl groups (—CO—R,) and alkoxy carbonyl or aryloxy carbonyl groups (—CO—O—R,), wherein R₁ is an aliphatic, substituted aliphatic, benzyl, substituted benzyl, aromatic or a substituted aromatic group. Specific examples of acyl groups include acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, phenyl-CO—, substituted phenyl-CO—, benzyl-CO— and (substituted benzyl)-CO—. Examples of alkoxy carbonyl and aryloxy carbonyl groups include CH₃-O—CO—, (ethyl)-O—CO—, n-propyl-O—CO—, isopropyl-O—CO—, n-butyl-O—CO—, sec-butyl-O—CO—, t-butyl-O—CO—, phenyl-O—CO—, substituted phenyl-O—CO— and benzyl-O—CO—, (substituted benzyl)-O—CO—. In order to facilitate the N-acylation, one to four glycine residues can be added to the N-terminus of the sequence. The carboxyl group at the C-terminus can be protected, for example, by an amide (i.e., the hydroxyl group at the C-terminus is replaced with —NH₂, —NHR₂ and —NR₂R₃) or ester (i.e. the hydroxyl group at the C-terminus is replaced with —OR₂). R₂ and R₃ are independently an aliphatic, substituted aliphatic, benzyl, substituted benzyl, aryl or a substituted aryl group. In addition, taken together with the nitrogen atom, R₂ and R₃ can form a C4 to C8 heterocyclic ring with from about 0-2 additional heteroatoms such as nitrogen, oxygen or sulfur. Examples of suitable heterocyclic rings include piperidinyl, pyrrolidinyl, morpholino, thiomorpholino or piperazinyl. Examples of C-terminal protecting groups include —NH₂, —NHCH₃, —N(CH₃)₂, —NH(ethyl), —N(ethyl)₂, N(methyl)(ethyl), —NH(benzyl), —N(C1-C4 alkyl)(benzyl), —NH(phenyl), —N(C1-C4 alkyl)(phenyl), —OCH₃, —O-(ethyl), —O-(n-propyl), —O-(n-butyl), —O-(isopropyl), —O-(sec-butyl), —O-(t-butyl), —O-benzyl and —O-phenyl.

An “amino acid residue” is a moiety found within a peptide and is represented by —NH—CHR—CO—, wherein R is the side chain of a naturally occurring amino acid. When referring to a moiety found within a peptide, the terms “amino acid residue” and “amino acid” are used in this application. An “amino acid residue analog” is either a peptidomimetic or is a D or L residue having the following formula: —NH—CHR—CO—, wherein R is an aliphatic group, a substituted aliphatic group, a benzyl group, a substituted benzyl group, an aromatic group or a substituted aromatic group and wherein R does not correspond to the side chain of a naturally-occurring amino acid. When referring to a moiety found within a peptide, the terms “amino acid residue analog” and “amino acid analog” are used interchangeably in this application. Amino acid analogs are well-known in the art; a large number of these analogs are commercially available.

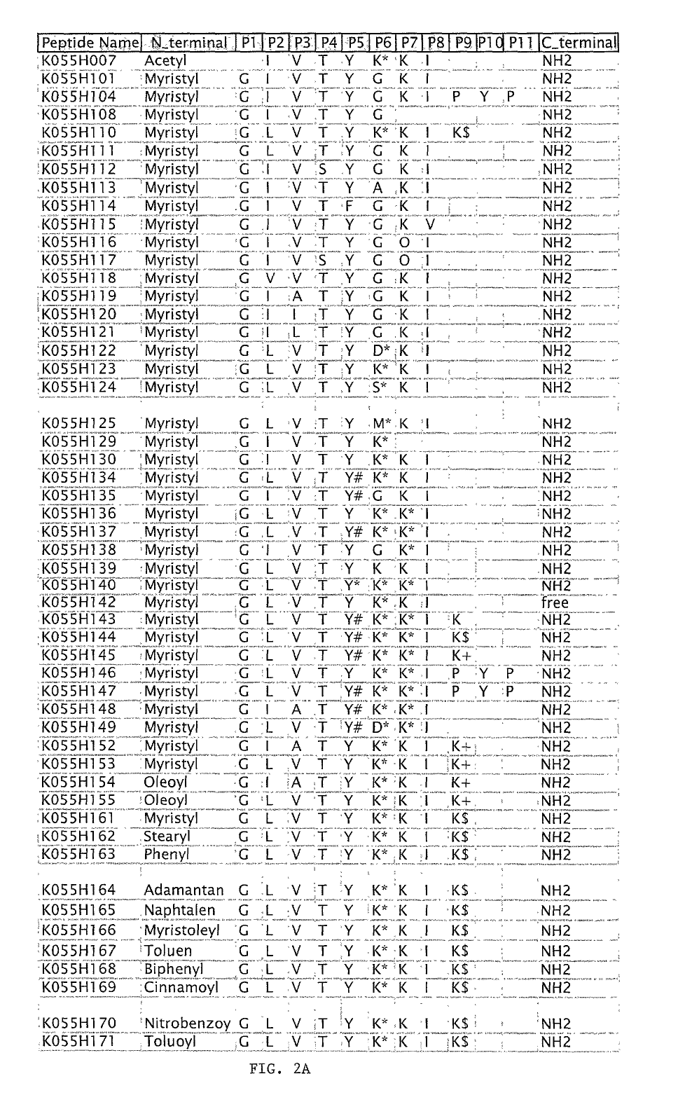

A “conservatively substituted amino acid”, also called a “conservatively substituted amino acid residue”, is an amino acid analog which, when substituted for a native (original) amino acid of the lyn peptides (shown herein as the peptides with SEQ ID numbers of FIG. 2 but without the N-terminal glycines or the denoted amino acid substitutions of these sequences) or is inserted as a spacer group in the amino acid sequence of the lyn peptides, does not severely alter the inhibitory activity of the peptide. A peptidomimetic of the naturally occurring amino acid, as well documented in the literature known to the skilled practitioner, also referred to as a “functional peptidomimetic” is an organic moiety which, when substituted for a native (original) amino acid of the lyn peptides or is inserted as a spacer group in the amino acid sequence of the lyn peptides, also does not severely alter the inhibitory activity of the peptide. The ability of such a lyn peptide derivative to affect the activities of prostate cancer cells expressing the lyn tyrosine kinase is not markedly different from the inhibitory ability of the native or original lyn peptide either when a conservatively substituted amino acid analog or functional peptidomimetic replaces a native amino acid of the native or original lyn peptide, or when a conservatively substituted amino acid analog or functional peptidomimetic is inserted in an amino acid sequence of a lyn peptide. Since such lyn peptide derivatives have inhibitory ability which is essentially the same as that of lyn peptides, these lyn peptide derivatives are also embodiments of this invention.

As used herein, aliphatic groups are straight chained, branched or cyclic C1-C8 hydrocarbons that are completely saturated, which contain one or two heteroatoms such as nitrogen, oxygen or sulfur and/or which contain one or more units of unsaturation. Aromatic groups are carbocyclic aromatic groups such as phenyl and naphthyl and heterocyclic aromatic groups such as imidazolyl, indolyl, thienyl, furanyl, pyridyl, pyranyl, pyrrolyl, oxazolyl, benzothienyl, benzofuranyl, quinolinyl, isoquinolinyl and acridintyl.

Suitable substituents on an aliphatic, aromatic or benzyl group include —OH, halogen (—Br, —Cl, —I and —F), —O(aliphatic, substituted aliphatic, benzyl, substituted benzyl, aryl or substituted aryl group), —CN, —NO₂, —COOH, —NH₂, —NH(aliphatic group, substituted aliphatic, benzyl, substituted benzyl, aryl or substituted aryl group), -N(aliphatic group, substituted aliphatic, benzyl, substituted benzyl, aryl or substituted aryl group)₂, —COO(aliphatic group, substituted aliphatic, benzyl, substituted benzyl, aryl or substituted aryl group), —CONH₂, —CONH(aliphatic, substituted aliphatic group, benzyl, substituted benzyl, aryl or substituted aryl group), —SH, —S(aliphatic, substituted aliphatic, benzyl, substituted benzyl, aromatic or substituted aromatic group) and —NH—C(32 NH)—NH₂. A substituted benzylic or aromatic group can also have an aliphatic or substituted aliphatic group as a substituent. A substituted aliphatic group can also have a benzyl, substituted benzyl, aryl or substituted aryl group as a substituent. A substituted aliphatic, substituted aromatic or substituted benzyl group can have one or more of these substituents.

Suitable substitutions for amino acid residues in the sequence of a lyn peptide include conservative substitutions which result in peptide derivatives which inhibit the activity of a lyn tyrosine kinase. Among the categories of amino acid substitutions, conservative substitutions are preferred in this invention. Particularly preferred, are amino acid substitutions where one, two or three amino acids are substituted by a conservative substitution. A “conservative substitution” is a substitution in which the substituting amino acid (naturally occurring or modified) has about the same steric and electronic properties as the amino acid being substituted. Thus, the substituting amino acid would have the same or a similar functional group in the side chain as the original amino acid.

A “conservative substitution” can also be achieved by utilizing a substituting amino acid that is identical to the amino acid being substituted except that a functional group in the side chain is functionalized with a suitable protecting group. Suitable protecting groups are described in Green and Wuts, “Protecting Groups in Organic Synthesis”, John Wiley and Sons, Chapters 5 and 7, 1991, the teachings of which are incorporated herein by reference. As with N-terminal and C-terminal protecting groups, preferred protecting groups are those which facilitate transport of the peptide into a cell, for example, by reducing the hydrophilicity and increasing the lipophilicity of the peptide, and which can be cleaved in vivo, either by hydrolysis or enzymatically, inside the cell. (Ditter et al., J. Pharm. Sci. 57:783 (1968); Ditter et al., J. Pharm. Sci. 57:828 (1968); Ditter et al., J. Pharm. Sci. 58:557 (1969); King et al., Biochemistry 26:2294 (1987); Lindberg et al., Drug Metabolism and Disposition 17:311 (1989); and Tunek et al., Biochem. Pharm. 37:3867 (1988), Anderson et al., Arch. Biochem. Biophys. 239:538 (1985) and Singhal et al., FASEB J. 1:220 (1987)). Hydroxyl protecting groups include esters, carbonates and carbamate protecting groups. Amine protecting groups include alkoxy and aryloxy carbonyl groups, as described above for N-terminal protecting groups. Carboxylic acid protecting groups include aliphatic, benzylic and aryl esters, as described above for C-terminal protecting groups. In one embodiment, the carboxylic acid group in the side chain of one or more glutamic acid or aspartic acid residue in a peptide of the present invention is protected, preferably with a methyl, ethyl, benzyl or substituted benzyl ester, more preferably as a benzyl ester.

Provided below are groups of naturally occurring and modified amino acids in which each amino acid in a group has similar electronic and steric properties. Thus, a conservative substitution is made by substituting an amino acid with another amino acid from the same group. It is to be understood that these groups are non-limiting, i.e. that there are additional modified amino acids which could be included in each group.

Group I includes leucine, isoleucine, valine, methionine, phenylalanine, serine, cysteine, threonine and modified amino acids having the following side chains: ethyl, n-butyl, —CH₂CH₂OH, —CH₂CH₂CH₂OH, —CH₂CHOHCH₃ and —CH₂SCH₃. Preferably, Group I includes leucine, isoleucine, valine and methionine.

Group II includes glycine, alanine, valine, serine, cysteine, threonine and a modified amino acid having an ethyl side chain. Preferably, Group II includes glycine and alanine.

Group III includes phenylalanine, phenylglycine, tyrosine, tryptophan, cyclohexylmethyl, and modified amino residues having substituted benzyl or phenyl side chains. Preferred substituents include one or more of the following: halogen, methyl, ethyl, nitro, methoxy, ethoxy and —CN. Preferably, Group III includes phenylalanine, tyrosine and tryptophan.

Group IV includes glutamic acid, aspartic acid, a substituted or unsubstituted aliphatic, aromatic or benzylic ester of glutamic or aspartic acid (e.g., methyl, ethyl, n-propyl iso-propyl, cyclohexyl, benzyl or substituted benzyl), glutamine, asparagine, CO—NH-alkylated glutamine or asparagine (e.g., methyl, ethyl, n-propyl and iso-propyl) and modified amino acids having the side chain -(CH₂)₃—COOH, an ester thereof (substituted or unsubstituted aliphatic, aromatic or benzylic ester), an amide thereof and a substituted or unsubstituted N-alkylated amide thereof. Preferably, Group IV includes glutamic acid, aspartic acid, glutamine, asparagine, methyl aspartate, ethyl aspartate, benzyl aspartate and methyl glutamate, ethyl glutamate and benzyl glutamate.

Group V includes histidine, lysine, arginine, N-nitroarginine, β-cycloarginine, g-hydroxyarginine, N-amidinocitruline and 2-amino-4-guanidinobutanoic acid, homologs of lysine, homologs of arginine and omithine. Preferably, Group V includes histidine, lysine, arginine, and ornithine. A homolog of an amino acid includes from 1 to about 3 additional methylene units in the side chain.

Group VI includes serine, threonine, cysteine and modified amino acids having C1-C5 straight or branched alkyl side chains substituted with —OH or —SH. Preferably, Group VI includes serine, cysteine or threonine.

In this invention any cysteine in the original sequence or subsequence can be replaced by a homocysteine or other sulfhydryl-containing amino acid residue or analog. Such analogs include lysine or beta amino alanine, to which a cysteine residue is attached through the secondary amine yielding lysine-epsilon amino cysteine or alanine-beta amino cysteine, respectively.

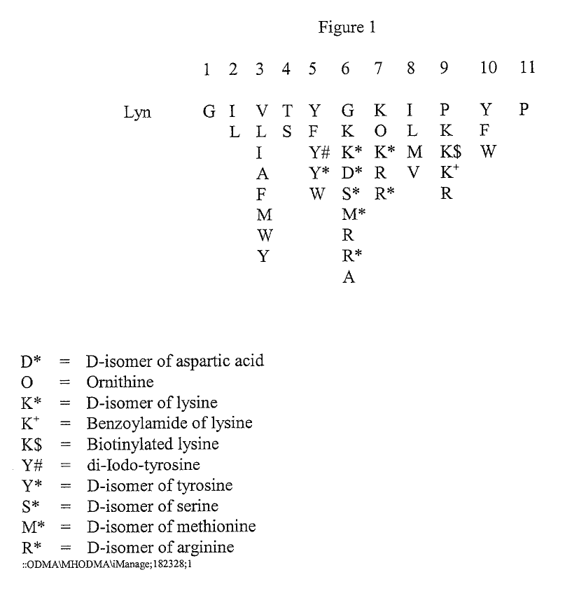

In another aspect, suitable substitutions for amino acid residues in the sequence of a lyn peptide include “severe substitutions” which result in peptide derivatives which inhibit the activity of a lyn tyrosine kinase. Severe substitutions which result in peptide derivatives that inhibit the activity of a lyn tyrosine kinase are much more likely to be possible in positions which are not highly conserved throughout the family of peptides than at positions which are highly conserved. FIG. 1 shows the consensus sequences of the six to eleven amino acids of the lyn peptides. Because D-amino acids have a hydrogen at a position identical to the glycine hydrogen side-chain, D-amino acids or their analogs can often be substituted for glycine residues.

A “severe substitution” is a substitution in which the substituting amino acid (naturally occurring or modified) has significantly different size, configuration and/or electronic properties compared with the amino acid being substituted. Thus, the side chain of the substituting amino acid can be significantly larger (or smaller) than the side chain of the amino acid being substituted and/or can have functional groups with significantly different electronic properties than the amino acid being substituted. Examples of severe substitutions of this type include the substitution of phenylalanine or cycohexylmethyl glycine for alanine, isoleucine for glycine, or —NH-CH[(—CH₂)₅—COOH]-CO—for aspartic acid. Alternatively, a functional group may be added to the side chain, deleted from the side chain or exchanged with another functional group. Examples of severe substitutions of this type include adding an amine or hydroxyl, carboxylic acid to the aliphatic side chain of valine, leucine or isoleucine, exchanging the carboxylic acid in the side chain of aspartic acid or glutamic acid with an amine or deleting the amine group in the side chain of lysine or ornithine. In yet another alternative, the side chain of the substituting amino acid can have significantly different steric and electronic properties from the functional group of the amino acid being substituted. Examples of such modifications include tryptophan for glycine, lysine for aspartic acid and -(CH₂)₄COOH for the side chain of serine. These examples are not meant to be limiting.

“Peptidomimetics” can be substituted for amino acid residues in the peptides of this invention. These peptidomimetics either replace amino acid residues or act as spacer groups within the peptides. The peptidomimetics often have steric, electronic or configurational properties similar to the replaced amino acid residues but such similarities are not necessarily required. The only restriction on the use of peptidomimetics is that the peptides retain their protein kinase modulating activity. Peptidomimetics are often used to inhibit degradation of the peptides by enzymatic or other degradative processes. The peptidomimetics can be produced by organic synthetic techniques. Examples of suitable peptidomimetics include D amino acids of the corresponding L amino acids, tetrazol (Zabrocki et al., J. Am. Chem. Soc. 110, 58755880 (1988)); isosteres of amide bonds (Jones et al., Tetrahedron Lett. 29, 3853-3856 (1988)); LL-3-amino-2-propenidone-6-carboxylic acid (LL-Acp) (Kemp et al., J. Org. Chem. 50, 5834-5838 (1985)). Similar analogs are shown in Kemp et al., Tetrahedron Lett. 29, 5081-5082 (1988) as well as Kemp et al., Tetrahedron Lett. 29, 5057-5060 (1988), Kemp et al., Tetrahedron Lett. 29, 4935-4938 (1988) and Kemp et al., J. Org. Chem. 54, 109-115 (1987). Other suitable peptidomimetics are shown in Nagai and Sato, Tetrahedron Lett. 26, 647-650 (1985); Di Maio et al., J. Chem. Soc. Perkin Trans., 1687 (1985); Kahn et al., Tetrahedron Lett. 30, 2317 (1989); Olson et al., J. Am. Chem. Soc. 112, 323-333 (1990); Garvey et al., J. Org. Chem. 56, 436 (1990). Further suitable peptidomimetics include hydroxy-1,2,3,4-tetrahydroisoquinoline-3-carboxylate (Miyake et al., J. Takeda Res. Labs 43, 53-76 (1989)); 1,2,3,4-tetrahydroisoquinoline-3carboxylate (Kazmierski et al., J. Am. Chem. Soc. 133, 2275-2283 (1991)); histidine isoquinolone carboxylic acid (HIC) (Zechel et al., Int. J. Pep. Protein Res. 43 (1991)); (2S, 3S)-methyl-phenylalanine, (2S, 3R)-methyl-phenylalanine, (2R, 3S)-methyl-phenylalanine and (2R, 3R)-methyl-phenylalanine (Kazmierski and Hruby, Tetrahedron Lett. (1991)).

The amino acid residues of the peptides can be modified by carboxymethylation, acylation, phosphorylation, glycosylation or fatty acylation. Ether bonds can be used to join the serine or threonine hydroxyl to the hydroxyl of a sugar. Amide bonds can be used to join the glutamate or aspartate carboxyl groups to an amino group on a sugar (Garg and Jeanloz, Advances in Carbohydrate Chemistry and Biochemistry, Vol. 43, Academic Press (1985); Kunz, Ang. Chem. Int. Ed. English 26, 294-308 (1987)). Acetal and ketal bonds can also be formed between amino acids and carbohydrates. Fatty acid acyl derivatives can be made, for example, by free amino group (e.g., lysine) acylation (Toth et al., Peptides: Chemistry, Structure and Biology, Rivier and Marshal, eds., ESCOM Publ., Leiden, 1078-1079 (1990)).

In one aspect, one, two or more of the amino acids in the sequence are modified with conservative substitutions; the substitutions can be in consensus positions, in non-consensus positions or in both. In another aspect, one, two or more of the amino acids in the sequence are modified with severe substitutions; the substitutions are preferably in non-consensus positions. FIG. 1 provides examples of conservative amino acid substitutions.

The peptides of this invention are most preferably modified by suitable protecting groups on the N-terminal amino acid such as acetyl, myristyl, stearyl, phenyl, adamantyl, naphthalyl, myristoleyl, toluyl, biphenyl, cinnamoyl, nitrobenzyl, benzoyl, furoyl, oleoyl, cyclohexyl, norboranyl, z-caproyl, ricinolelyl, and palmitoyl substituents. Particularly preferred are the addition of glycine, with one of those protecting groups substituted at the N-terminal amine of the glycine, to the N-terminus of the peptide. The C-terminal amino acid can be modified by suitable protecting groups such as by amidation. The interior amino acids of the peptides can have substituents added without loss of effectiveness and, in many instances, with enhanced effectiveness as well as, often, longer biological half-lives because they are not recognized or degraded by degradative enzymes or processes that exist in the individual or the prostate cancer cells. These substituents include benzylamide groups on lysine, biotinylation of lysine and di-iodination of tyrosine. In addition, the D-isomer of any amino acid can be substituted for the natural L-isomer. This is particularly true for the lysine residues.

In this invention, particularly preferred peptides are those labeled as K055H007 (SEQ ID NO: l); K055H101 (SEQ ID NO:2); K055H104 (SEQ ID NO:3); K055H108 (SEQ ID NO:4); K055H110 (SEQ ID NO:5); K055H111 (SEQ ID NO:6); K055H112 (SEQ ID NO:7); K055H113 (SEQ ID NO:8); K055H114 (SEQ ID NO:9); K055H115 (SEQ ID NO:10); K055H116 (SEQ ID NO:101); K055H117 (SEQ ID NO:12); K055H118 (SEQ ID NO:13); K055H119 (SEQ ID NO:14); K055H120 (SEQ ID NO:15); K055H121 (SEQ ID NO:16); K055H122 (SEQ ID NO:17); K055H123 (SEQ ID NO:18); K055H124 (SEQ ID NO:19); K055H125 (SEQ ID NO:20); K055H129 (SEQ ID NO:21); K055H130 (SEQ ID NO:22); K055H134 (SEQ ID NO:23); K055H135 (SEQ ID NO:24); K055H136 (SEQ ID NO:25); K055H137 (SEQ ID NO:26); K055H138 (SEQ ID NO:27); K055H139 (SEQ ID NO:28); K055H140 (SEQ ID NO:29); K055H142 (SEQ ID NO:30); K055H143 (SEQ ID NO:31); K055H144 (SEQ ID NO:32); K055H145 (SEQ ID NO:33); K055H146 (SEQ ID NO:34); K055H147 (SEQ ID NO:35); K055H148 (SEQ ID NO:36); K055H149 (SEQ ID NO:37); K055H152 (SEQ ID NO:38); K055H153 (SEQ ID NO:39); K055H154 (SEQ ID NO:40); K055H155 (SEQ ID NO:41); K055H161 (SEQ ID NO:42); K055H162 (SEQ ID NO:43); K055H163 (SEQ ID NO:44); K055H164 (SEQ ID NO:45); K055H165 (SEQ ID NO:46); K055H166 (SEQ ID NO:47); K055H167 (SEQ ID NO:48); K055H168 (SEQ ID NO:49); K055H169 (SEQ ID NO:50); K055H170 (SEQ ID NO:51); K055H171 (SEQ ID NO:52); K055H172 (SEQ ID NO:53); K055H173 (SEQ ID NO:54); K055H174 (SEQ ID NO:55); K055H175 (SEQ ID NO:56); K055H176 (SEQ ID NO:57); K055H177 (SEQ ID NO:58); K055H300 (SEQ ID NO:59); K055H301 (SEQ ID NO:60); K055H302 (SEQ ID NO:61); K055H304 (SEQ ID NO:62); K055H305 (SEQ ID NO:63); K055H306 (SEQ ID NO:64); K055H307 (SEQ ID NO:65); K055H801 (SEQ ID NO:66); K055H902 (SEQ ID NO:67); K055H908 (SEQ ID NO:68); K055H910 (SEQ ID NO:69); K055H911 (SEQ ID NO:70); K055H912 (SEQ ID NO:71); K055H919 (SEQ ID NO:72); K055H923 (SEQ ID NO:73); K055H925 (SEQ ID NO:74), as specified in FIG. 2.

The N-terminus and/or C-terminus of these peptides can be modified, as described above and as shown in FIG. 2. The N-terminal of these peptides is substituted and the C-terminal is amidated. Other protecting groups for amides and carboxylic acids can be used, as described above. Optionally, one or both protecting groups can be omitted. The peptides may be linear or cyclic. Also included are peptides having the sequence of: K055H007 (SEQ ID NO: 1); K055H101 (SEQ ID NO:2); K055H104 (SEQ ID NO:3); K055H108 (SEQ ID NO:4); K055H110 (SEQ ID NO:5); K055H1 11 (SEQ ID NO:6); K055H112 (SEQ ID NO:7); K055H113 (SEQ ID NO:8); K055H114 (SEQ ID NO:9); K055H115 (SEQ ID NO:10); K055H116 (SEQ ID NO:1 1); K055H117 (SEQ ID NO: 12); K055H118 (SEQ ID NO: 13); K055H119 (SEQ ID NO:14); K055H120 (SEQ ID NO:15); K055H121 (SEQ ID NO:16); K055H122 (SEQ ID NO:17); K055H123 (SEQ ID NO:18); K055H124 (SEQ ID NO:19); K055H125 (SEQ ID NO:20); K055H129 (SEQ ID NO:21); K055H130 (SEQ ID NO:22); K055H134 (SEQ ID NO:23); K055H135 (SEQ ID NO:24); K055H136 (SEQ ID NO:25); K055H137 (SEQ ID NO:26); K055H138 (SEQ ID NO:27); K055H139 (SEQ ID NO:28); K055H140 (SEQ ID NO:29); K055H142 (SEQ ID NO:30); K055H143 (SEQ ID NO:31); K055H144 (SEQ ID NO:32); K055H145 (SEQ ID NO:33); K055H146 (SEQ ID NO:34); K055H147 (SEQ ID NO:35); K055H148 (SEQ ID NO:36); K055H149 (SEQ ID NO:37); K055H152 (SEQ ID NO:38); K055H153 (SEQ ID NO:39); K055H154 (SEQ ID NO:40); K055H155 (SEQ ID NO:41); K055H161 (SEQ ID NO:42); K055H162 (SEQ ID NO:43); K055H163 (SEQ ID NO:44); K055H164 (SEQ ID NO:45); K055H165 (SEQ ID NO:46); K055H166 (SEQ ID NO:47); K055H167 (SEQ ID NO:48); K055H168 (SEQ ID NO:49); K055H169 (SEQ ID NO:50); K055H170 (SEQ ID NO:51); K055H171 (SEQ ID NO:52); K055H172 (SEQ ID NO:53); K055H173 (SEQ ID NO:54); K055H174 (SEQ ID NO:55); K055H175 (SEQ ID NO:56); K055H176 (SEQ ID NO:57); K055H177 (SEQ ID NO:58); K055H300 (SEQ ID NO:59); K055H301 (SEQ ID NO:60); K055H302 (SEQ ID NO:61); K055H304 (SEQ ID NO:62); K055H305 (SEQ ID NO:63); K055H306 (SEQ ID NO:64); K055H307 (SEQ ID NO:65); K055H801 (SEQ ID NO:66); K055H902 (SEQ ID NO:67); K055H908 (SEQ ID NO:68); K055H910 (SEQ ID NO:69); K055H911 (SEQ ID NO:70); K055H912 (SEQ ID NO:71); K055H919 (SEQ ID NO:72); K055H923 (SEQ ID NO:73); K055H925 (SEQ ID NO:74) as specified in FIG. 2, with the proviso that any one or two of the amino residues in the peptide can vary, being replaced by any naturally occurring amino acid or analog thereof.

The present invention also includes cyclic peptides having amino acid sequences corresponding to a modified sequence of the lyn peptides. These cyclic peptides inhibit the activity of lyn tyrosine kinase.

A “cyclic peptide” refers, in one instance, to a peptide or peptide derivative in which a ring is formed by the formation of a peptide bond between the nitrogen atom at the N-terminus and the carbonyl carbon at the C-terminus.

“Cyclized” also refers to the forming of a ring by a covalent bond between the nitrogen at the N-terminus of the compound and the side chain of a suitable amino acid in the peptide, preferably the side chain of the C-terminal amino acid. For example, an amide can be formed between the nitrogen atom at the N-terminus and the carbonyl carbon in the side chain of an aspartic acid or a glutamic acid. Alternatively, the peptide or peptide derivative can be cyclized by forming a covalent bond between the carbonyl at the C-terminus of the compound and the side chain of a suitable amino acid in the peptide, preferably the side chain of the N-terminal amino acid. For example, an amide can be formed between the carbonyl carbon at the C-terminus and the amino nitrogen atom in the side chain of a lysine or an ornithine. Additionally, the peptide or peptide derivative can be cyclized by forming an ester between the carbonyl carbon at the C-terminus and the hydroxyl oxygen atom in the side chain of a serine or a threonine.

“Cyclized” also refers to forming a ring by a covalent bond between the side chains of two suitable amino acids in the peptide, preferably the side chains of the two terminal amino acids. For example, a disulfide can be formed between the sulfur atoms in the side chains of two cysteines. Alternatively, an ester can be formed between the carbonyl carbon in the side chain of, for example, a glutamic acid or an aspartic acid, and the oxygen atom in the side chain of, for example, a serine or a threonine. An amide can be formed between the carbonyl carbon in the side chain of, for example, a glutamic acid or an aspartic acid, and the amino nitrogen in the side chain of, for example, a lysine or an omithine.

In addition, a peptide or peptide derivative can be cyclized with a linking group between the two termini, between one terminus and the side chain of an amino acid in the peptide or peptide derivative, or between the side chains to two amino acids in the peptide or peptide derivative. Suitable linking groups are disclosed in Lobl et al., WO 92/00995 and Chiang et al., WO 94/15958, the teachings of which are incorporated into this application by reference.

It can be readily determined whether a peptide or peptide derivative inhibits the activity of lyn tyrosine kinase by incubating the peptide or peptide derivative with prostate cancer cells which have one or more cellular activities controlled by the lyn tyrosine kinase. The cells are incubated with the peptide or peptide derivative to produce a test mixture under conditions suitable for assessing the activity of the lyn tyrosine kinase. The activity of the lyn tyrosine kinase is assessed and compared with a suitable control, e.g., the activity of the same cells incubated under the same conditions in the absence of the peptide or peptide derivative. A lesser activity of the lyn tyrosine kinase in the test mixture compared with the control indicates that the test peptide or peptide derivative inhibits the activity of the lyn tyrosine kinase.

Conditions suitable for assessing lyn tyrosine kinase activity include conditions suitable for assessing a cellular activity or function under control of the lyn tyrosine kinase. Generally, a cellular activity or function can be assessed when the cells are exposed to conditions suitable for cell growth, including a suitable temperature (for example, between about 30° C. to about 42° C.) and the presence of the suitable concentrations of nutrients in the medium (e.g., amino acids, vitamins, growth factors).

Generally, the activity of the lyn tyrosine kinase in the test mixture is assessed by making a quantitative measure of the cellular activity which the lyn tyrosine kinase controls. The cellular activity can be, for example, cell proliferation. Lyn tyrosine kinase activity is assessed by measuring cellular proliferation, for example, by comparing the number of cells present after a given period of time with the number of cells originally present.

It is to be understood that the assay described hereinabove for determining whether a peptide or peptide derivative inhibits a cellular activity or function under the control of lyn tyrosine kinase can be performed with cells other than those specifically described herein.

The present invention is directed to methods of inhibiting the activity of a lyn tyrosine kinase in a subject. A “subject” is preferably a human, but can also be animals in need of treatment, e.g., veterinary animals (e.g., dogs, cats, and the like), farm animals (e.g., cows, pigs, horses, chickens and the like) and laboratory animals (e.g., rats, mice, guinea pigs and the like).

A “therapeutically effective amount” is the quantity of compound which results in an improved clinical outcome as a result of the treatment compared with a typical clinical outcome in the absence of the treatment. An “improved clinical outcome” results in the individual with the disease experiencing fewer symptoms or complications of the disease, including a longer life expectancy, as a result of the treatment. With respect to cancer, an “improved clinical outcome” includes a longer life expectancy. It can also include slowing or arresting the rate of growth of a tumor, causing a shrinkage in the size of the tumor, a decreased rate of metastasis and/or improved quality of life (e.g., a decrease in physical discomfort or an increase in mobility).

The amount of peptide or peptide derivative administered to the individual will depend on the type and severity of the disease and on the characteristics of the individual, such as general health, age, sex, body weight and tolerance to drugs. The skilled artisan will be able to determine appropriate dosages depending on these and other factors. Typically, a therapeutically effective amount of the peptide or peptide derivative can range from about 1 mg per day to about 1000 mg per day for an adult. Preferably, the dosage ranges from about 1 mg per day to about 100 mg per day.

The peptide and peptide derivatives of the present invention are preferably administered parenterally. Parenteral administration can include, for example, systemic administration, such as by intramuscular, intravenous, subcutaneous, or intraperitoneal injection. Peptides or peptide derivatives which resist proteolysis can be administered orally, for example, in capsules, suspensions or tablets. The peptide or peptide derivative can also be administered by inhalation or insufflation or via a nasal spray. The peptide or peptide derivative can be administered to the individual in conjunction with an acceptable pharmaceutical carrier as part of a pharmaceutical composition for treating the diseases discussed above. Suitable pharmaceutical carriers may contain inert ingredients which do not interact with the peptide or peptide derivative. Standard pharmaceutical formulation techniques may be employed such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., Controlled Release of Biological Active Agents, John Wiley and Sons, 1986).

The peptide and peptide derivatives of the present invention have many utilities other than as a therapeutic agent. Some of these uses are discussed in the following paragraphs.

The lyn-derived peptides of the present invention can be useful in the preparation of specific antibodies against lyn tyrosine kinase. Suitable antibodies can be raised against a lyn peptide by conjugating the peptide to a suitable carrier, such as keyhole limpet hemocyanin or serum albumin; polyclonal and monoclonal antibody production can be performed using any suitable technique. A variety of methods have been described (see e.g., Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer 1994), Ausubel, F. M. et al., Eds., (John Wiley & Sons: New York, NY), Chapter 11, (1991)). Generally, a hybridoma can be produced by fusing a suitable immortal cell line (e.g., a myeloma cell line such as SP2/0) with antibody producing cells. The antibody producing cell, preferably those of the spleen or lymph nodes, can be obtained from animals immunized with the antigen of interest. The fused cells (hybridomas) can be isolated using selective culture conditions, and cloned by limiting dilution. Cells which produce antibodies with the desired specificity can be selected by a suitable assay (e.g., ELISA).

Antibodies, including monoclonal antibodies, against lyn peptides have a variety of uses. For example, those against or reactive with lyn tyrosine kinase and preferably which bind specifically to lyn tyrosine kinase, can be used to identify and/or sort cells exhibiting that kinase on the cell surface (e.g., by means of fluorescence activated cell sorting or histological analyses). Monoclonal antibodies specific for lyn tyrosine kinase can also be used to detect and/or quantitate the kinase expressed on the surface of a cell or present in a sample (e.g., in an ELISA). Alternatively, the antibodies can be used to determine if an intracellular lyn tyrosine kinase is present in the cytoplasm of the cell. A lysate of the cell is generated (for example, by treating the cells with sodium hydroxide (0.2 N) and sodium dodecyl sulfate (1%) or with a non-ionic detergent like NP-40, centrifugating and separating the supernatant from the pellet), and treated with anti-lyn peptide antibody specific for lyn tyrosine kinase. The lysate is then analyzed, for example, by Western blotting or immunoprecipitation for complexes between lyn tyrosine kinase and antibody. Anti-lyn peptide antibodies can be utilized for the study of the intracellular distribution (compartmentalization) of lyn tyrosine kinase under various physiological conditions via the application of conventional immunocytochemistry such as immunofluorescence, immunoperoxidase technique and immunoelectron microscopy, in conjunction with the specific anti-lyn peptide antibody. Antibodies reactive with the lyn peptides are also useful to detect and/or quantitate the lyn tyrosine kinase in a sample, or to purify the lyn tyrosine kinase (e.g., by immunoaffinity purification).

The lyn-derived peptides of the present invention can also be used to identify ligands which interact with lyn tyrosine kinase and which inhibit the activity of lyn tyrosine kinase. For example, an affinity column can be prepared to which a lyn peptide is covalently attached, directly or via a linker. This column, in turn, can be utilized for the isolation and identification of specific ligands which bind the lyn peptide and which will also likely bind the lyn tyrosine kinase. The ligand can then be eluted from the column, characterized and tested for its ability to inhibit lyn tyrosine kinase function.

Peptide sequences in the compounds of the present invention may be synthesized by solid phase peptide synthesis (e.g., t-BOC or F-MOC) method, by solution phase synthesis, or by other suitable techniques including combinations of the foregoing methods. The t-BOC and F-MOC methods, which are established and widely used, are described in Merrifield, J. Am. Chem. Soc. 88:2149 (1963); Meienhofer, Hormonal Proteins and Peptides, C. H. Li, Ed., Academic Press, 1983, pp. 48-267; and Barany and Merrifield, in The Peptides, E. Gross and J. Meienhofer, Eds., Academic Press, New York, 1980, pp. 3-285. Methods of solid phase peptide synthesis are described in Merrifield, R. B., Science, 232: 341 (1986); Carpino, L. A. and Han, G. Y., J. Org. Chem., 37: 3404 (1972); and Gauspohl, H. et al., Synthesis, 5: 315 (1992)). The teachings of these references are incorporated herein by reference.

Methods of cyclizing compounds having peptide sequences are described, for example, in Lobl et al., WO 92/00995, the teachings of which are incorporated herein by reference. Cyclized compounds can be prepared by protecting the side chains of the two amino acids to be used in the ring closure with groups that can be selectively removed while all other side-chain protecting groups remain intact. Selective deprotection is best achieved by using orthogonal side-chain protecting groups such as allyl (OAI) (for the carboxyl group in the side chain of glutamic acid or aspartic acid, for example), allyloxy carbonyl (Aloc) (for the amino nitrogen in the side chain of lysine or ornithine, for example) or acetamidomethyl (Acm) (for the sulfhydryl of cysteine) protecting groups. OAI and Aloc are easily removed by Pd° and Acm is easily removed by iodine treatment.

The invention is illustrated by the following examples which are not intended to be limiting in any way.

### EXAMPLE 1

### Preparation of Lyn Peptides

The compounds of this invention can be synthesized utilizing a 430A Peptide Synthesizer from Applied Biosystems using F-Moc technology according to manufacturer's protocols. Other suitable methodologies for preparing peptides are known to person skilled in the art. See e.g., Merrifield, R. B., Science, 232: 341 (1986); Carpino, L. A., Han, G. Y., J Org. Chem., 37: 3404 (1972); Gauspohl, H., et al., Synthesis, 5: 315 (1992)). The teachings of which are incorporated herein by reference.

Rink Amide Resin [4(2′, 4′ Dimethoxyphenyl-FMOC amino methyl) phenoxy resin] was used for the synthesis of C-amidated peptides. The alpha-amino group of the amino acid was protected by an FMOC group, which was removed at the beginning of each cycle by a weak base, 20% piperidine in N-methylpyrrolidone (NMP). After deprotection, the resin was washed with NMP to remove the piperidine. In situ activation of the amino acid derivative was performed by the FASTMOC Chemistry using HBTU (2(1-benzotriazolyl-1-yl)-,1,1,3,3-tetramethyluronium) dissolved in HOBt (1-hydroxybenzotriazole) and DMF (dimethylformamide). The amino acid was dissolved in this solution with additional NMP. DIEA (diisopropylethylamine) was added to initiate activation. Alternatively, the activation method of DCC (dicycbohexylcarbodiimide) and HOBL was utilized to form an HOBt active ester. Coupling was performed in NMP. Following acetylation of the N-terminus (optional), TFA (trifluoroacetic acid) cleavage procedure of the peptide from the resin and the side chain protecting groups was applied using 0.75 g crystalline phenol; 0.25 ml EDT (1,2ethandithiol); 0.5 ml thioanisoie; 0.5 ml D.I. H₂O; 10 ml TFA.

### EXAMPLE 2

### Inhibition of Proliferation of Prostate Cancer Cells In Vitro by Incubation with Lyn Peptides

Human prostate cancer cell lines PC3 and DU145 were obtained from the American Type Culture Collection (ATCC No. 1435-CRL and 81-HTB). These cell lines were grown in RPMI 1640 medium supplemented with penicillin (100 U/ml), streptomycin (100 μg/ml), glutamine (2 mM) and 10% endotoxin free bovine cell serum (Hyclone).

A suspension of the cells at 25×10³ cells/ml was prepared in the above described culture medium and distributed 0.160 ml per well (about 4000 cells/well) in the wells of 96 well, flat bottom, tissue culture microtiter plates.

A series of lyn peptide stock solutions were prepared by diluting a 10 mM solution of the lyn peptide in 100% DMSO with phosphate buffered saline (PBS) containing 0.1% bovine serum albumin (BSA) to a concentration of 400 μM. These solutions were labeled DMSO. In many instances, 40 μl of the 10 mM lyn peptide in DMSO solution was mixed with 160 μl of 2M NH₄HCO₃ and heated for 40 minutes at 100° C. The resultant solution was then diluted to 400 μM in PBS containing 0.1% BSA. These lyn peptide stock solutions were labeled “tbi”. The concentration of lyn peptide in each stock solution was adjusted to nine times the desired concentration of the lyn peptide in the assay mixture. 0.020 ml of each lyn peptide stock solution was added to the corresponding wells about 2 hours after prostate cancer cell addition, with six replicates for each concentration. In addition, BSA solution with no added lyn peptide was used as a control. The plates were incubated for 72-80 hours at 37° C. in a 10% CO₂ humidified incubator.

The plates were labeled and the medium discarded. Each plate was then washed one time with PBS (0.20 ml/well). The wells were then fixed by washing with 100% ethanol. (0.20 ml/well for 5 minutes). The ethanol was removed and the wells dried completely. Alternatively, the wells were fixed with 4% formaldehyde PBS (PBS buffered with 10% formalin from Fisher Scientific; Catalog No. HC200-1) (0.12 ml/well) for at least 30 minutes. Fixing with formaldehyde enhances the O.D. compared with ethanol fixation.

The wells were washed one time with borate buffer (0.1M, pH 8.5). Freshly filtered 1% methylene blue solution (0.60 ml/well) was then added to the wells and incubated for 10 minutes at room temperature. The wells were then washed five times with tap water, after which the wells were dried completely. 0.20 ml/well of 0.1 N HCl was added to extract the color. After overnight extraction, the O.D. was read at 630 nm to determine the number of cells per well. The procedure for counting cells is described in greater detail in Oliver et al. J Cell Sci., 92: 513 (1989), the teachings of which are incorporated herein by reference.

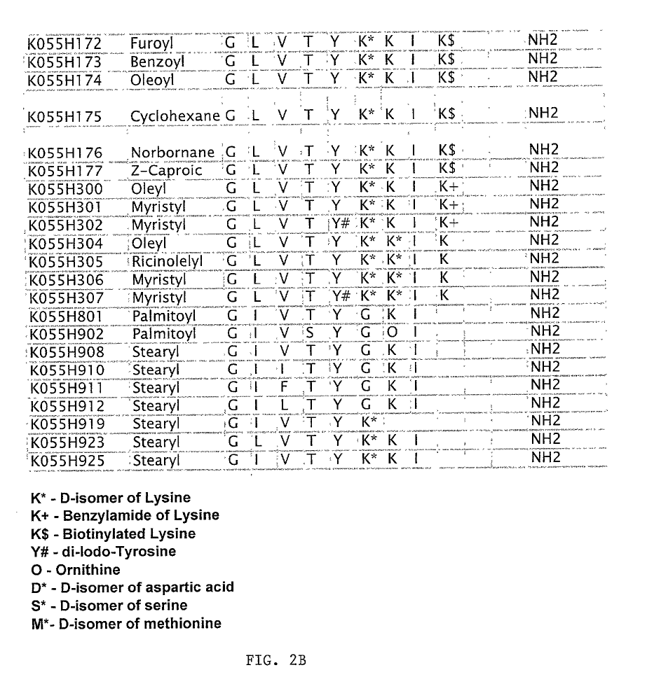

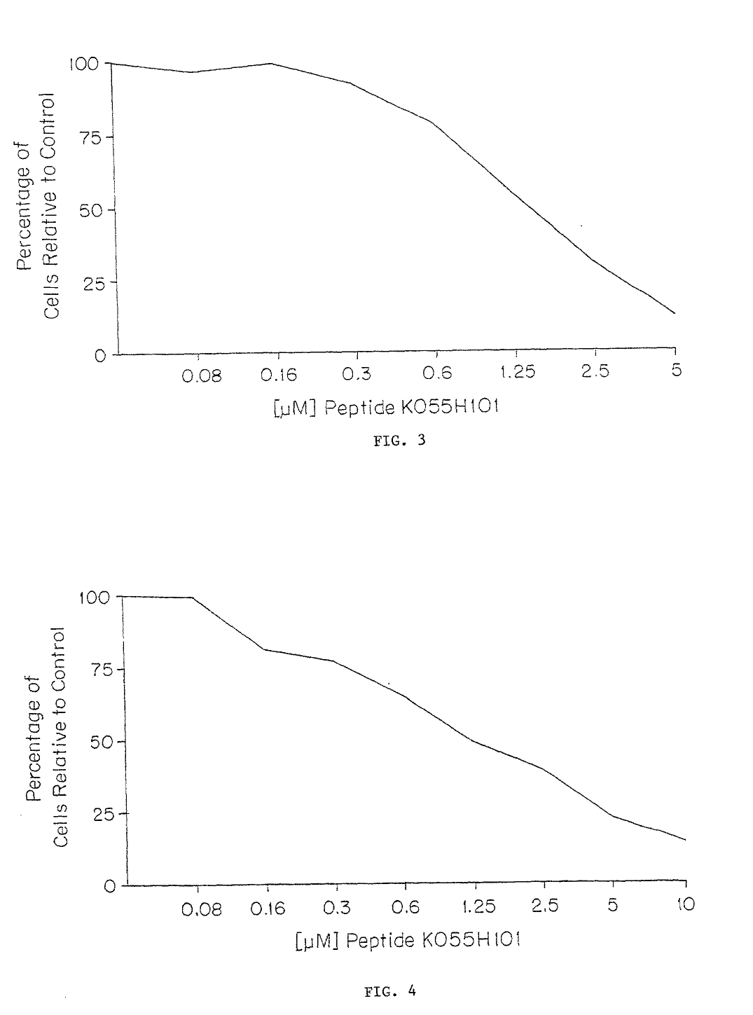

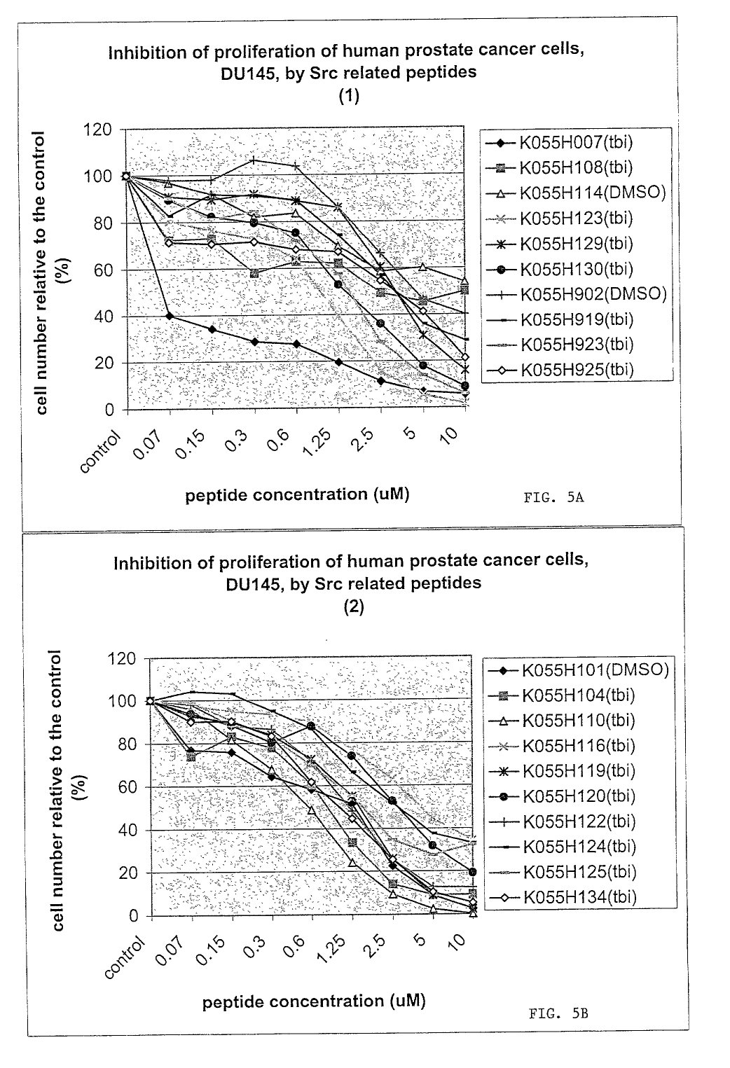

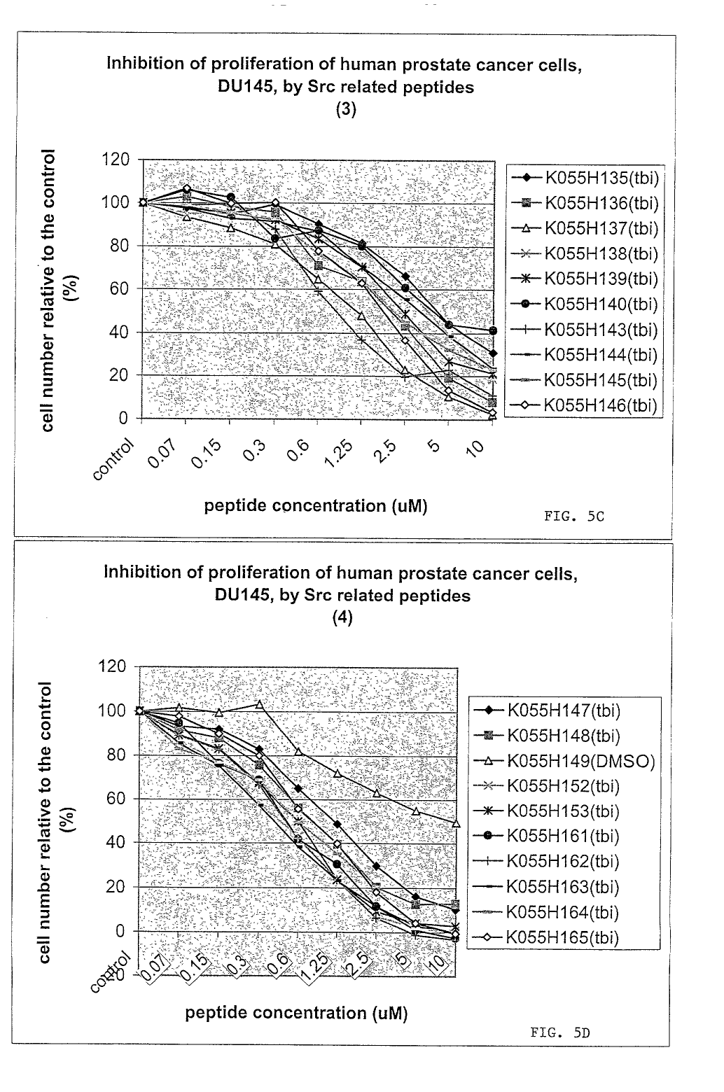

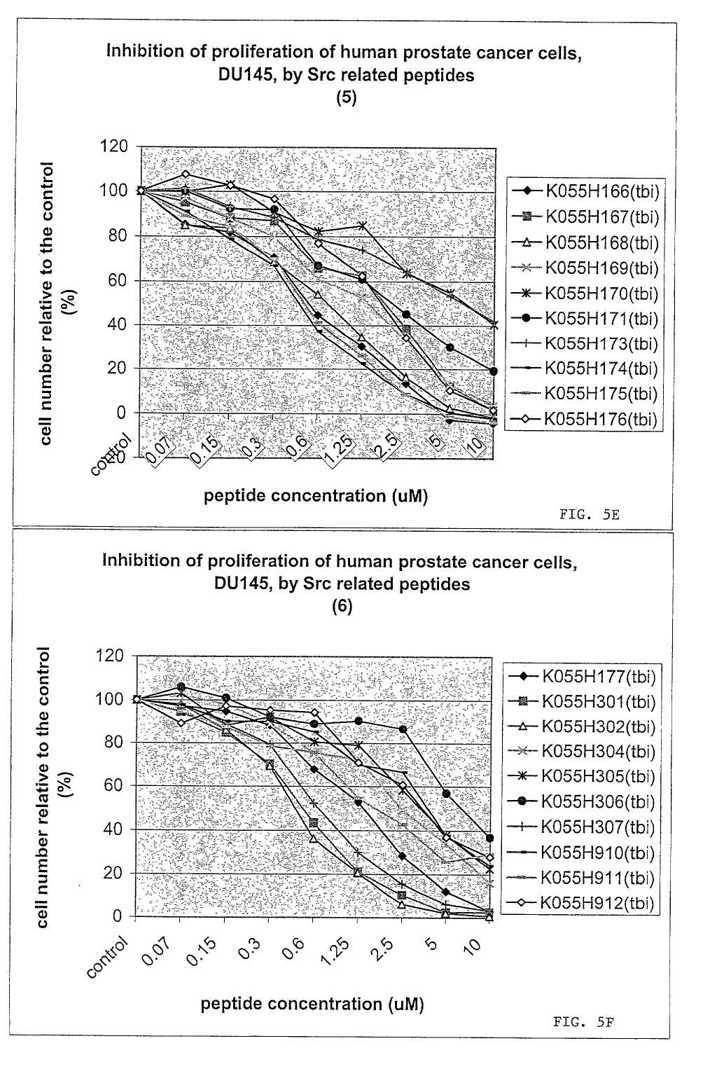

The results are shown in FIGS. 3, 4 and 5A-5F. The data in these Figures show that the lyn peptides inhibit growth of prostate cancer cells. As can be seen in FIG. 3, peptide K055H101 (tbi) inhibited the proliferation of DU145 cells relative to control at concentrations greater than about 0.3 μM. As can be seen in FIG. 4, peptide K055H101 (tbi) inhibited the proliferation of cells from the PC3 cell line at concentrations greater than 0.16 μM. As shown in FIGS. 5A-5F, for some peptides, the inhibition of prostate cancer cell growth becomes noticeable at peptide concentrations of 0.6 μM. For other peptides, significant inhibition occurs at concentrations at least as low as 0.07 μM.

### EXAMPLE 3

### Prostate Cancer Tumor Shrinkage in Nude Mice

The hormone-refractory human prostate cancer cell line, DU-145, was grown in RPMI-1640 culture medium with 10% fatal calf serum plus antibodies (see Example 2). The DU-145 cells were harvested and injected subcutaneously into male nude mice strain CD 1 of about 6-7 weeks of age, 4×10⁶ cells per mouse. After about 6 to 8 weeks, when the tumors became palpable, treatment of these mice was started by intraperitoneal (i.p.) injection of 40 mg/kg of a peptide solution containing the K055H137 (tbi) peptide. The peptide solutions were prepared by taking 40 μl of a 50 mM peptide in DMSO solution, mixing it with 160 μl of 2 M NH₄HCO₃ and heating it for 40 minutes at 100° C. The resultant solution was diluted 1:3 in 2M Hepes (pH 7.0) prior to injection. These injections were continued weekly thereafter. Control mice received i.p. injections of vehicle only. Tumor volume was measured twice a week.

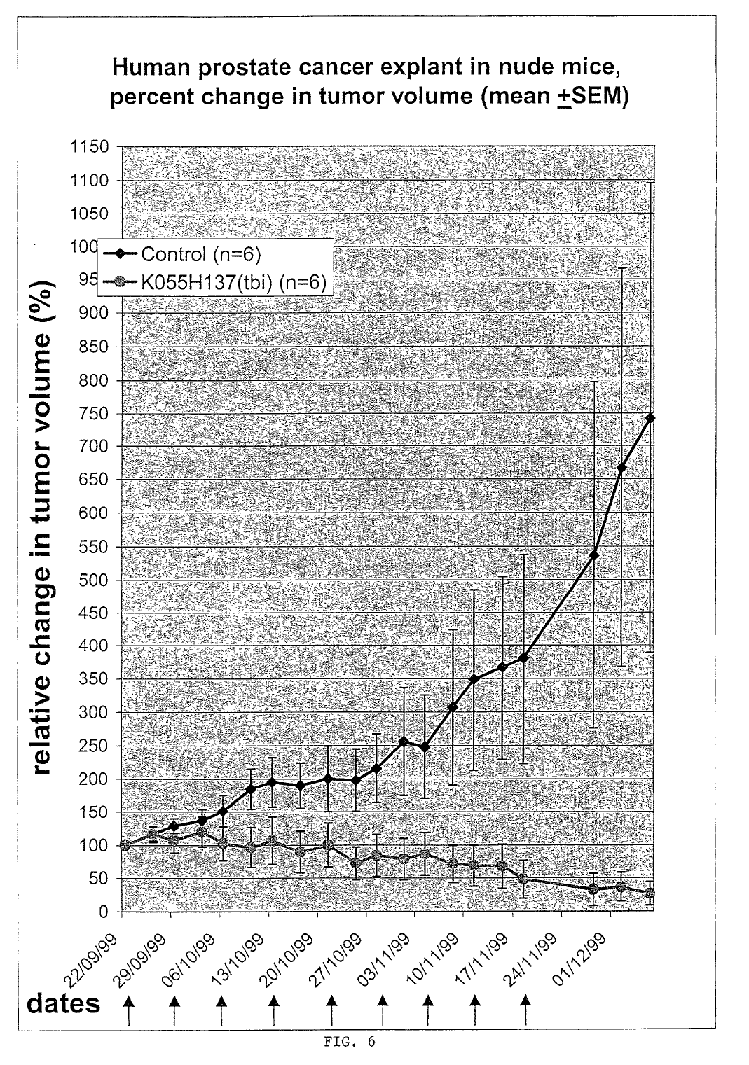

The results of the lyn peptide injections on prostate cancer tumor development is shown in FIG. 6. It can be seen that the tumor diminishes in size with time when lyn peptide injections are administered. By contrast, the tumors in control animals grow exponentially over the same time period. Clearly, the lyn peptide has an effect on the prostate cancer tumor beyond slowing tumor growth. The administration of the lyn peptide actually causes the prostate cancer tumors to diminish in size. By 11 weeks after the first lyn peptide injection, the tumors are one quarter the size they initially were.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treatment of prostate cancer in an individual, in need thereof, comprising administration of an inhibitor of lyn tyrosine kinase to said individual, wherein said administration results in a reduction or stasis of said prostate cancer when compared to the occurrence of said prostate cancer without said administration.

2. The method of claim 1 wherein said inhibitor is selected from the group consisting of peptides, antibodies, anti-sense nucleic acids, negative dominant Lyn genes and small organic molecules.

3. The method of claim 2 wherein said small organic molecule is a pyrazolo pyrimidine-type inhibitor.

4. The method of claim 2 wherein said inhibitor is a lyn-derived peptide.

5. The method of claim 4 wherein said lyn-derived peptide is selected from the group consisting of K055H007 (SEQ ID NO:1); K055H101 (SEQ ID NO:2);
K055H104 (SEQ ID NO:3); K055H108 (SEQ ID NO:4); K055H 110 (SEQ ID NO:5); K055H111 (SEQ ID NO:6); K055H112 (SEQ ID NO:7); K055H113 (SEQ ID NO:8); K055H114 (SEQ ID NO:9); K055H115 (SEQ ID NO:10);
K055H116(SEQ ID NO:11);K055H117(SEQ ID NO:12);K055H118(SEQ ID NO:13); K055H119 (SEQ ID NO:14); K055H120 (SEQ ID NO:15); K055H121 (SEQ ID NO:16); K055H122 (SEQ ID NO:17); K055H123 (SEQ ID NO:18); K055H124 (SEQ ID NO:19); K055H125 (SEQ ID NO:20); K055H129 (SEQ ID NO:21); K055H130 (SEQ ID NO:22); K055H134 (SEQ ID NO:23); K055H135 (SEQ ID NO:24); K055H136 (SEQ ID NO:25); K055H137 (SEQ ID NO:26); K055H138 (SEQ ID NO:27); K055H139 (SEQ ID NO:28); K055H140 (SEQ ID NO:29); K055H142 (SEQ ID NO:30); K055H143 (SEQ ID NO:31); K055H144 (SEQ ID NO:32); K055H145 (SEQ ID NO:33); K055H146 (SEQ ID NO:34); K055H147 (SEQ ID NO:35); K055H148 (SEQ ID NO:36); K055H149 (SEQ ID NO:37); K055H152 (SEQ ID NO:38); K055H153 (SEQ ID NO:39); K055H154 (SEQ ID NO:40); K055H155 (SEQ ID NO:41); K055H161 (SEQ ID NO:42); K055H162 (SEQ ID NO:43); K055H163 (SEQ ID NO:44); K055H164 (SEQ ID NO:45); K055H165 (SEQ ID NO:46); K055H166 (SEQ ID NO:47); K055H167 (SEQ ID NO:48); K055H168 (SEQ ID NO:49); K055H169 (SEQ ID NO:50); K055H170 (SEQ ID NO:51); K055H171 (SEQ ID NO:52); K055H172 (SEQ ID NO:53); K055H173 (SEQ ID NO:54); K055H174 (SEQ ID NO:55); K055H175 (SEQ ID NO:56); K055H176 (SEQ ID NO:57); K055H177 (SEQ ID NO:58); K055H300 (SEQ ID NO:59); K055H301 (SEQ ID NO:60); K055H302 (SEQ ID NO:61); K055H304 (SEQ ID NO:62); K055H305 (SEQ ID NO:63); K055H306 (SEQ ID NO:64); K055H307 (SEQ ID NO:65); K055H801 (SEQ ID NO:66); K055H902 (SEQ ID NO:67); K055H908 (SEQ ID NO:68); K055H910 (SEQ ID NO:69); K055H911 (SEQ ID NO:70); K055H912 (SEQ ID NO:71); K055H919 (SEQ ID NO:72); K055H923 (SEQ ID NO:73); K055H925 (SEQ ID NO:74) as specified in FIG. 2.

6. A method of inhibiting the growth of prostate cancer cells comprising administration of an inhibitor of lyn tyrosine kinase to said prostate cancer cells, whereby said administration results in an inhibition of growth of said prostate cancer cells.

7. The method of claim 6 wherein said inhibitor is selected from the group consisting of peptides, antibodies, anti-sense nucleic acids, negative dominant Lyn genes and small organic molecules.

8. The method of claim 7 wherein said small organic molecule is a pyrazolo pyrimidine-type inhibitor.

9. The method of claim 6 wherein said inhibitor is a lyn-derived peptide.

10. The method of claim 9 wherein said lyn-derived peptide is selected from the group consisting of K055H007 (SEQ ID NO:1); K055H101 (SEQ ID NO:2 K055H007 (SEQ ID NO:1); K055H101 (SEQ ID NO:2); K055H104 (SEQ ID NO:3); K055H108 (SEQ ID NO:4); K055Hl 10 (SEQ ID NO:5); K055H111 (SEQ ID NO:6); K055H112 (SEQ ID NO:7); K055H113 (SEQ ID NO:8); K055H114 (SEQ ID NO:9); K055H115 (SEQ ID NO:10); K055H116 (SEQ ID NO:11); K055H117 (SEQ ID NO:12); K055H118 (SEQ ID NO:13); K055H119 (SEQ ID NO: 14); K055H120 (SEQ ID NO:15); K055H121 (SEQ ID NO:16); K055H122 (SEQ ID NO:17); K055H123 (SEQ ID NO:18); K055H124 (SEQ ID NO:19); K055H125 (SEQ ID NO:20); K055H129 (SEQ ID NO:21); K055H130 (SEQ ID NO:22); K055H134 (SEQ ID NO:23); K055H135 (SEQ ID NO:24); K055H136 (SEQ ID NO:25); K055H137 (SEQ ID NO:26); K055H138 (SEQ ID NO:27); K055H139 (SEQ ID NO:28); K055H140 (SEQ ID NO:29); K055H142 (SEQ ID NO:30); K055H143 (SEQ ID NO:31); K055H144 (SEQ ID NO:32); K055H145 (SEQ ID NO:33); K055H146 (SEQ ID NO:34); K055H147 (SEQ ID NO:35); K055H148 (SEQ ID NO:36); K055H149 (SEQ ID NO:37); K055H152 (SEQ ID NO:38); K055H153 (SEQ ID NO:39); K055H154 (SEQ ID NO:40); K055H155 (SEQ ID NO:41); K055H161 (SEQ ID NO:42); K055H162 (SEQ ID NO:43); K055H163 (SEQ ID NO:44); K055H164 (SEQ ID NO:45); K055H165 (SEQ ID NO:46); K055H166 (SEQ ID NO:47); K055H167 (SEQ ID NO:48); K055H168 (SEQ ID NO:49); K055H169 (SEQ ID NO:50); K055H170 (SEQ ID NO:51); K055H171 (SEQ ID NO:52); K055H172 (SEQ IUD NO:53); K055H173 (SEQ ID NO:54); K055H174 (SEQ ID NO:55); K055H175 (SEQ ID NO:56); K055H176 (SEQ ID NO:57); K055H177 (SEQ ID NO:58); K055H300 (SEQ ID NO:59); K055H301 (SEQ ID NO:60); K055H302 (SEQ ID NO:61); K055H304 (SEQ ID NO:62); K055H305 (SEQ ID NO:63); K055H306 (SEQ ID NO:64); K055H307 (SEQ ID NO:65); K055H801 (SEQ ID NO:66); K055H902 (SEQ ID NO:67); K055H908 (SEQ ID NO:68); K055H910 (SEQ ID NO:69); K055H911 (SEQ ID NO:70); K055H912 (SEQ ID NO:71); K055H919 (SEQ ID NO:72); K055H923 (SEQ ID NO:73); K055H925 (SEQ ID NO:74), as specified in FIG. 2.

11. A peptide having an amino acid sequence selected from the group consisting of: K055H007 (SEQ ID NO:1); K055H101 (SEQ ID NO:2 K055H007 (SEQ ID NO:1); K055H101 (SEQ ID NO:2); K055H104 (SEQ ID NO:3); K055H108 (SEQ ID NO:4); K055H110 (SEQ ID NO:5); K055H111 (SEQ ID NO:6); K055H112 (SEQ ID NO:7); K055H113 (SEQ ID NO:8); K055H114 (SEQ ID NO:9); K055H115 (SEQ ID NO:10); K055H116 (SEQ ID NO: 1); K055H117 (SEQ ID NO:12); K055H118 (SEQ ID NO:13); K055H119 (SEQ ID NO:14); K055H120 (SEQ ID NO:15); K055H121 (SEQ ID NO:16); K055H122 (SEQ ID NO:17); K055H123 (SEQ ID NO:18); K055H124 (SEQ ID NO:19); K055H125 (SEQ ID NO:20); K055H129 (SEQ ID NO:21); K055H130 (SEQ ID NO:22); K055H134 (SEQ ID NO:23); K055H135 (SEQ ID NO:24); K055H136 (SEQ ID NO:25); K055H137 (SEQ ID NO:26); K055H138 (SEQ ID NO:27); K055H139 (SEQ ID NO:28); K055H140 (SEQ ID NO:29); K055H142 (SEQ ID NO:30); K055H143 (SEQ ID NO:31); K055H144 (SEQ ID NO:32); K055H145 (SEQ ID NO:33); K055H146 (SEQ ID NO:34); K055H147 (SEQ ID NO:35); K055H148 (SEQ ID NO:36); K055H149 (SEQ ID NO:37); K055H152 (SEQ ID NO:38); K055H153 (SEQ ID NO:39); K055H154 (SEQ ID NO:40); K055H155 (SEQ ID NO:41); K055H161 (SEQ ID NO:42); K055H162 (SEQ ID NO:43); K055H163 (SEQ ID NO:44); K055H164 (SEQ ID NO:45); K055H165 (SEQ ID NO:46); K055H166 (SEQ ID NO:47); K055H167 (SEQ ID NO:48); K055H168 (SEQ ID NO:49); K055H169 (SEQ ID NO:50); K055H170 (SEQ ID NO:51); K055H171 (SEQ ID NO:52); K055H172 (SEQ IUD NO:53); K055H173 (SEQ ID NO:54); K055H174 (SEQ ID NO:55); K055H175 (SEQ ID NO:56); K055H176 (SEQ ID NO:57); K055H177 (SEQ ID NO:58); K055H300 (SEQ ID NO:59); K055H301 (SEQ ID NO:60); K055H302 (SEQ ID NO:61); K055H304 (SEQ ID NO:62); K055H305 (SEQ ID NO:63); K055H306 (SEQ ID NO:64); K055H307 (SEQ ID NO:65); K055H801 (SEQ ID NO:66); K055H902 (SEQ ID NO:67); K055H908 (SEQ ID NO:68); K055H910 (SEQ ID NO:69); K055H911 (SEQ ID NO:70); K055H912 (SEQ ID NO:71); K055H919 (SEQ ID NO:72); K055H923 (SEQ ID NO:73); and K055H925 (SEQ ID NO:74).
